(19) United States
(12) Patent Application Publication
(10) Pub. No.: US 2025/0009467 A1
(43) Pub. Date: Jan. 9, 2025

(54) LAPAROSCOPIC SURGICAL TOOL TIP HOLDER

(71) Applicant: Modular Surgical, Inc., Cupertino, CA (US)

(72) Inventors: Bryce E. PETERSON, Hayward, CA (US); David W. PEREZ, Menlo Park, CA (US); Mitchell K. CARROLL, San Francisco, CA (US); Amir BELSON, Sayvon (IL)

(21) Appl. No.: 18/767,774

(22) Filed: Jul. 9, 2024

Related U.S. Application Data
(63) Continuation of application No. PCT/US2023/060250, filed on Jan. 6, 2023.
(60) Provisional application No. 63/298,157, filed on Jan. 10, 2022.

(51) Int. Cl. A61B 90/50; A61B 17/00

(52) U.S. Cl. CPC A61B 90/50; A61B 2017/00464; A61B 2017/00557

(57) ABSTRACT

A tool tip holder includes a deployment mechanism and several tool carriers coupled to the deployment mechanism. Each tool carrier has a tool access end and a base end, and the deployment mechanism is adapted to selectively reconfigure the plurality of tool carriers from a linear or other elongated arrangement into a parallel cluster. The deployment mechanism can be a spring attachment structure that links the tool carriers and folds them into a parallel cluster when released from constraint. Alternatively, the deployment mechanism may be an inflatable structure that arranges the tool carrier into a parallel cluster when inflated.

### CROSS-REFERENCE TO RELATED APPLICATIONS

This application is a continuation of International Application No. PCT/US2023/060250, filed Jan. 6, 2023, which claims the benefit of U.S. Provisional No. 63/298,157, filed Jan. 10, 2022, the entire content of which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

### 1. Field of the Invention

The present invention relates generally to medical devices and methods. More particularly, the present invention relates to surgical tool tip holders for use in laparoscopic and endoscopic procedures where a single tool shaft can be used with multiple interchangeable surgical tool tips.

Laparoscope, endoscopic and other minimally invasive surgical procedures have been widely adopted and have replaced many open surgical procedures. Laparoscopic procedures are performed within body cavities, such as the abdomen or thorax, and rely on introducing a shaft of a surgical tool through tissue via a small incision, trocar or other entry site which provide access into the cavity. In laparoscopic procedures, the shaft manipulates a tool or end effector to perform a surgical protocol while viewing the surgical field within the cavity using a laparoscopic camera. One drawback in performing laparoscopic and other minimally invasive procedures is the need to frequently exchange tools through a limited number of access points.

It has been proposed to increase the efficiency of laparoscopic and other minimally invasive procedures by introducing a plurality of surgical tool tips into the surgical field, such as an insufflated abdomen or a thorax, and subsequently exchanging tools on a single or limited number of tool shafts, thus avoiding the need to remove and exchange complete tools through the access points. Such surgical tools exchange systems and procedures are described, for example, in commonly owned U.S. Patent Publication Nos. 2013/0150871 and 2015/0216515, the full disclosures of which are incorporated herein by reference.

U.S. Patent Publication No. 2015/0216515 describes the use of a tool tip holder, referred to as a cassette, for introducing, holding and facilitating the exchange of multiple tools in an insufflated abdomen, thorax or other surgical field. The tool cassette includes up to five articulated segments, most or all of which carry deployable tool tip holders and one of which may be an external attachment segment. The tool cassettes are introduced through an incision or port in a straightened configuration, curved for stabilization, and either placed on a tissue surface or pulled against an upper wall of the thorax using a manipulator attached to the external attachment segment.

While functional and of significant benefit in performing laparoscopic and other minimally invasive procedures, the designs disclosed in U.S. Patent Publication Nos. 2013/0150871 and 2015/0216515, are first generation designs, and it would be advantageous to provide additional and alternative designs having improved ease-of use, design simplicity, reliability, compactness, and the like. At least some of these objectives will be met by the inventions described and claimed hereinafter.

### 2. Background Art

U.S. Patent Publications 2013/0150871 and 2015/0216515 have been described above. See also U.S. Pat. Nos. 5,925,002; 6,309,397; 8,747,394; 8,858,538; 7,492,116; as well as U.S. Publication Nos. 2003/0114731; 2005/0043718; U.S. 2005/0165449; U.S. 2005/0209607; U.S. 2006/0020287; U.S. 2006/0041273; U.S. 2007/0198000; U.S. 2008/0021274; U.S. 2008/0108871; U.S. 2008/0147096; US. 2008/0167672; U.S. 2008/0275480; U.S. 2009/0005638; U.S. 2009/0005635; U.S. 2009/0182193; U.S. 2010/0016855; U.S. 2010/0057078; U.S. 2010/0188493; U.S. 2011/0087267; U.S. 2012/0083826; U.S. 2012/01322450; U.S. 2013/0066304; and U.S. 2013/0211196.

### SUMMARY OF THE INVENTION

In a first aspect, the present invention provides a tool tip holder or cassette comprising a deployment mechanism and a plurality of tool carriers. The tool carriers are coupled to the deployment mechanism, and each tool carrier has a tool access end and a base end. The deployment mechanism is adapted to selectively reconfigure the plurality of tool carriers from a linear or otherwise straightened arrangement useful for introduction to and removal of the tip holder from the surgical filed into a parallel cluster useful for accessing tool tips in each tool carrier.

By “parallel cluster,” it is meant that longitudinal axes of the plurality of tool carriers are held in a parallel arrangement, typically in a symmetric pattern about a center axis. Usually, but not necessarily, the tool carriers have access ends and base ends which are aligned in planes perpendicular to the center axis so that the parallel clusters have a generally cylindrical arrangement with the tool access ends present in a circular periphery at one end of the cylinder and the base ends present in a circular periphery at the other end of the cylinder. While the cross-sectional peripheries will usually be circular, in other instances they could be ellipsoidal, polygonal, and the like.

The linear or straightened arrangement of the tool carriers has a low profile which is particularly suited for minimally invasive introduction of the tool tip holder assembly through an access port into an abdominal or thoracic surgical field. The tool tip holder can be reconfigured into the parallel cluster arrangement after introduction to the surgical field, and the parallel cluster arrangement is particularly suited for removal and return of individual surgical tool tips from and to individual tool carriers of the tool tip holder while present in the surgical field. The deployment mechanism will typically allow the tool tip holder assembly to be reconfigured remotely by the surgeon using a manipulator, an inflator, or other tool introduced concurrently with or after the tool tip holder has been placed, as will be described in greater detail below.

The tool tip holder of the present invention will typically further comprise one or more surgical working tips removably placed in at least some of the plurality of tool carriers. The surgical tool tips will usually be placed into the tool carriers prior to introduction of the tool tip holder into the patient, but in some instances, it may be possible to introduce and/or exchange surgical tool tips after the procedure has commenced. The individual tool carriers will usually comprise tubular bodies, such cylindrical bodies, but will often have flat surfaces or facets to facilitate forming the parallel clusters, as described in more detail below.

In a preferred aspect, the tool tip holders or cassettes of the present invention may further comprise a hub segment configured to occupy a center region of the parallel cluster when the tool carriers are unconstrained, i.e., free to self-assemble or be assembled into the parallel cluster as described in more detail below. The hub is typically further configured to removably attach to a manipulator. The manipulator may be pre-attached to the hub of the tool tip holder or may be introduced separately into the surgical field before or after the tool tip holder has been introduced but prior to the reconfiguration of the initial linear arrangement tool carriers into the desired parallel cluster.

In some examples, the tool carriers and the hub may each comprise one or more magnetic elements, where the magnetic elements on the hub are configured to attract the magnetic elements on the tool carriers to hold or stabilize the tool carriers in their parallel cluster configuration while allowing limited displacement to facilitate removal and return of tool tips from the tool carriers.

In other examples, one or more of the tool carriers may comprise a mechanical latch mechanism, such as a hook, lock-and-key arrangement, snap, or the like, configured to selectively engage the hub to maintain all the tool carriers in the cluster formation while allowing limited displacement of one or more tool carriers (typically a pair of tool carriers joined at the top by a spring or other coupling element) to facilitate removal and return of tool tips from the tool carriers. In preferred instances, both magnetic elements and a latch mechanism will be used, where the magnets provide an initial alignment, and the latch stabilizes the cluster configuration as the tool carriers are displaced and surgical tools removed and returned to individual carrier.

In some examples, individual tool carriers or groups of tools carriers may be configured to be displaced away from the hub to facilitate access to a tool carried by the tool carrier. For example, the individual tool carriers or groups of tools carriers may be configured to be displaced away from the hub by engagement with a pusher, such as a wire, ribbon, or other elongate, typically flexible element that can be advanced over or through the shaft.

In some examples, the tool tip cassette may further comprise a tether attached to the hub and configured to temporarily support the parallel cluster of tool carriers prior to capture by a tool tip cassette deployment and support tool.

In some instances, the deployment and support tool may comprise a reciprocatable tip. The reciprocatable tip will typically be configured to pierce tissue when distally advanced. In other instances, the reciprocatable tip may be conjured to lock, unlock, manipulate, and otherwise interact with the tool tip holder as described in detail herein below. For example, the reciprocatable tip may be configured to engage and release the tool tip cassette when said cassette in its parallel cluster arrangement.

In many instances, the hub comprises flat faces or facets disposed axially about its periphery, with each flat face being configured to engage a first flat surface on one of the tool carriers. For example, the plurality of tool carriers may comprise any one of (1) three tool carriers with axial facets having a 120° spacing, (2) four tool carriers with axial facets having a 90° spacing, (3) five tool carriers with axial facets having a 72° spacing, (4) six tool carriers with axial facets having a 60° spacing, (5) seven tool carriers with axial facts having a 51.4° spacing, and (6) eight tool carriers with spring axial facets having a 45° spacing. Often, each tool carrier will comprise two additional flat surfaces arranged symmetrically on each side of first flat surface and configured to nest with adjacent tool carriers when the tool carriers are in the parallel cluster formation.

The deployment mechanism may have any one of the variety of structures. For example, a self-assembling deployment mechanism may comprise spring elements which link the tool access ends and the base ends of adjacent tool carriers, wherein the spring elements on adjacent tool carriers are typically circumferentially offset so that the spring elements cause the tool carriers to fold into the parallel cluster when unconstrained. In preferred instances, the springs members may comprise two legs having a U-shape when unconstrained, wherein a first leg is attached to a base end or a tool access end of one tool carrier and a second leg is attached to a base end or a tool access end of an adjacent tool carrier.

A hub used in combination with the spring elements is typically be attached to the base end of a first tool carrier by a U-shaped spring configured so that the plurality of tool carriers fold into the parallel cluster about a periphery of the hub when released from constraint.

Such self-assembling tool tip holders will typically require some structure, functionality, or other means configured apply a radial constraint to maintain the tool carriers in a generally straightened or otherwise elongated configuration for delivery to the patient, typically comprising a straight over tube having a passage for receiving, straightening, and delivering the plurality of tool carriers to a surgical site. While the use of a straight over tube is generally preferred, in some instance the over tube could have a small curvature or other nonlinearity so long as a deviation would not interfere with introduction and/or removal of the tool tip holder in its elongated configuration.

In other instances, the deployment mechanism may comprise an inflatable structure, where the plurality of tool carriers can be straightened or axially elongated, i.e., aligned generally axially, when the inflatable structure is deflated for delivery and removal of the tool tip holders. The inflatable structure will have a geometry selected and configured to arrange into a parallel cluster when the inflatable structure is inflated at a deployment site in the patient.

In specific instances, the inflatable structure may comprise a ring or disc. For example, the inflatable structure may comprise an open ring, often with a C-shape, with spokes or fingers projecting radially inwardly, wherein the tool tip holders are attached to radially inward ends of the spokes or fingers.

In a still further aspect, the present invention provides a tool tip delivery system including any of the tool tip holders described previously together with a support/release tool. The support/release tool comprises a shaft having a proximal end, a distal end, and a handle at the proximal end of the shaft. The distal end of the shaft is configured to detachably engage the tool tip holder, typically a manipulator segment that is attached to the hub.

In some instances, the support/release tool and the manipulator segment are configured to selectively align and misalign magnets in the hub with magnets in the tool carriers of the tool tip holder to form and release the parallel cluster arrangement. For example, the magnets within the hub may be mounted to axially translate, and the support/release tool and/or manipulator segment may comprise a pusher or puller to move the hub magnets in and out of alignment with the tool carrier magnets. In a specific example, the manipulator segment comprises a puller cable, and the hub magnet is spring-mounted so that puller cable can apply tension to align the hub and tool tip holder magnets while the cassette manipulator remains attached to the hub. The support/release tool includes an actuator, such as a translatable rod, which engages the manipulator segment and allows a user to shift the magnets alignment as desired.

In specific examples, the manipulator segment and hub may be connected via a universal joint, where the universal joint is typically located at the distal end of the manipulator segment and the proximal end of the hub.

In some instances, the tool tip cassette delivery system of the present invention may further comprise a first interlock configured to prevent disengagement of the surgical tool tip from the shaft when the tip is removed from the tool carrier.

In some instances, the tool tip cassette delivery system of the present invention may further comprise a second interlock configured to prevent loss of the surgical tool tip from the tool carrier when the tool tip is not engaged with the tool driver.

In yet another aspect, the present invention provides a method for delivering surgical tools to a surgical working space. The method may comprise providing a tool tip cassette including a plurality of tool carriers joined end-to-end. A tool tip cassette is percutaneously introduced to the surgical working site while the plurality of tool carriers is in a linearized arrangement. The plurality of tool carriers is reconfigured from the linearized arrangement into a parallel cluster while in the surgical working space, allowing surgical tools to be retrieved from and returned to individual tool carriers while the plurality of tool carriers remains in the parallel cluster arrangement in the surgical working space.

In some instances, the methods may further comprise reconfiguring the plurality of tool carriers from the parallel cluster arrangement to the linearized arrangement while in the surgical working space. The tool tip cassette may be percutaneously removed from the surgical working site while the plurality of tool carriers is in the linearized arrangement.

In some instances, reconfiguring the plurality of tool carriers from the linearized arrangement into the parallel cluster may comprise releasing the tool tip cassette from constraint.

In some instances, reconfiguring the plurality of tool carriers from the linearized arrangement into the parallel cluster may comprise applying a force to the tool tip cassette to affect the reconfiguration.

In some instances, the methods may further comprise capturing the tool tip cassette with a tool tip cassette deployment and support tool after the plurality of tool carriers have assumed the parallel cluster configuration.

Typically, the tool tip cassette deployment and support tool are percutaneously introduced through a percutaneous passage separate from that used to introduce the tool tip cassette.

In some instances, the methods may further comprise manipulating a surgical tool driver to engage and remove a surgical tool from an individual tool carrier after the plurality of tool carriers have assumed the parallel cluster configuration.

Typically, the surgical tool driver is percutaneously introduced through a percutaneous passage separate from that used to introduce the tool tip cassette and/or the tool tip cassette deployment and support tool.

### BRIEF DESCRIPTION OF THE DRAWINGS

FIG. 1 is an illustration of a first embodiment of parallel cluster of articulated tool carriers constructed in accordance with the principles of the present invention.

FIG. 2 illustrates a manipulator tool of the present invention detachably secured to a hub of the tool carrier cluster.

FIG. 3 is a perspective view showing the manipulator tool and hub of the present invention connected to the tool carrier cluster.

FIG. 4 illustrates the tool carrier cluster of the present invention in a straightened or elongated configuration where individual tool carriers are joined end-to-end in a manner suitable for percutaneous introduction into a patient.

FIG. 5 is an exploded view of a pair of adjacent tool carriers showing a spring attachment mechanism which folds the tool carriers as they are released from constraint when delivered to the patient within the principles of the present invention.

FIG. 6 is a schematic illustration of an arrangement of tool carriers surrounding a hub showing the relative positions of magnets in a stabilizing magnetic array when the tool carriers are formed into a parallel cluster in accordance with the principles of the present invention.

FIGS. 7A-7E illustrate the steps in releasing and self-folding of a tool cluster array in accordance with the principles of the present invention.

FIG. 8 is an illustration of a second embodiment of parallel cluster of tool carriers on an inflatable structure in accordance with the principles of the present invention.

FIGS. 9 and 10 illustrate the tool carrier embodiment of FIG. 8 shown with the inflatable structure in a partially inflated and deflated configuration, respectively.

FIGS. 11 and 12 illustrate the inflatable support structure of FIGS. 9 and 10 in greater detail in a deflated and inflated configuration, respectively.

FIG. 13 illustrates a magnetic hub that can be used to help stabilize the tool carrier embodiment of FIG. 8 during use.

FIGS. 14-16 illustrate a canned mechanism for selectively releasing the tool carriers from the magnetic hub of FIG. 13.

FIG. 17 illustrates a tool tip cassette deployment and support tool having a shaft with a proximal end and a distal end and a deployment handle at the proximal end.

FIG. 17A is a cross-sectional view taken along line 17A-17A in FIG. 17.

FIG. 18 is an enlarged view of the deployment handle of the tool tip cassette deployment and support tool of FIG. 17.

FIGS. 19A to 19B are detailed views of the deployment handle of the tool tip cassette deployment and support tool of FIG. 17 showing different operational settings.

FIGS. 20A to 20D are enlarged views of the distal end of the shaft of the tool tip cassette deployment and support tool of FIG. 17.

FIGS. 21A and 21B show the tool tip cassette of the present invention in its parallel cluster configuration as it is being engaged and captured by the distal end of the tool tip cassette deployment and support tool of FIG. 17.

FIGS. 22A and 22B show the tool tip cassette of the present invention in its parallel cluster configuration with a latch mechanism in an open configuration where the individual tool carriers are not latched to the hub (FIG. 22A) and a closed configuration where the individual tool carriers are latched to the hub (FIG. 22B).

FIGS. 23A and 23B are detailed views of the latch mechanism of FIGS. 22A and 22B shown in an open configuration (FIG. 23A) and a closed configuration (FIG. 23B).

FIGS. 24A and 24B are detailed views showing a pusher radially deflecting different tool carriers from a tool carrier cassette cluster to facilitate removal of surgical tools from the carriers.

FIG. 25 illustrates a surgical tool driver having a shaft with a proximal end and a distal end and a deployment handle at the proximal end.

FIGS. 26A to 26D are detailed views of the deployment handle of the surgical tool driver tool of FIG. 25 showing different operational settings.

FIGS. 27A to 27E are enlarged views of the distal end of the shaft of the surgical tool driver of FIG. 25 shown in different configurations.

FIGS. 28A to 28C show the distal tip of the surgical tool driver engaging and removing a surgical tool from a tool carrier.

FIGS. 29A and 29B show the surgical tool being actuated by the surgical tool driver.

FIGS. 30A and 30B show a safety lock mechanism which prevents the surgical tool from being accidentally dropped from the tool driver when the surgical tool is outside the tool carrier.

FIGS. 31A and 31B show a tool retention mechanism holds the surgical tool in the tool carrier and which is actuated by the surgical tool driver to allow the tool to be removed from the carrier.

### DETAILED DESCRIPTION OF THE INVENTION

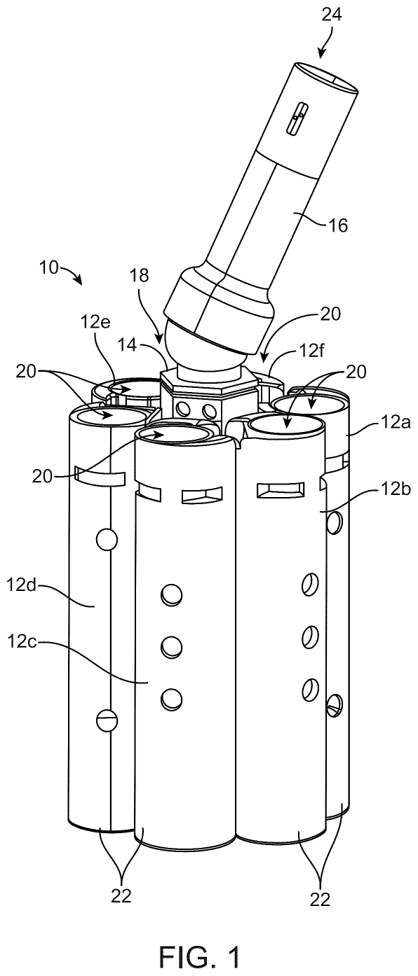

As shown in FIG. 1, a tool carrier array 10 is formed as a parallel cluster of individual tool carriers 12a-12f. While six individual tool carriers are illustrated, the number of carriers in the array may vary anywhere from 2 to 10, or even greater. The individual tool carriers 12 in the array are configured so that they may be brought together, or “clustered,” about a central axis, typically defined by an elongate hub 14. When so clustered, tool access ends 20 of each of the tool carriers 12 are arranged in a circular or other regular pattern adjacent to the top of the hub 14. The tool access ends are open or otherwise configured to allow access to surgical tool tips therein. The specific structure of the carriers is not critical and does not form part of the present invention. Exemplary designs useful together with the tool carrier arrays of the present invention can be seen in commonly owned, published patent application US2015/0216515, full disclosure of which has previously been incorporated herein by reference. Base ends 22 of each tool carrier 12 are similarly arranged in a circular or other regular pattern adjacent to a lower end of the hub. As will be described in greater detail below, the hub 14 is arranged to interface with and stabilize the individual tool carriers 12.

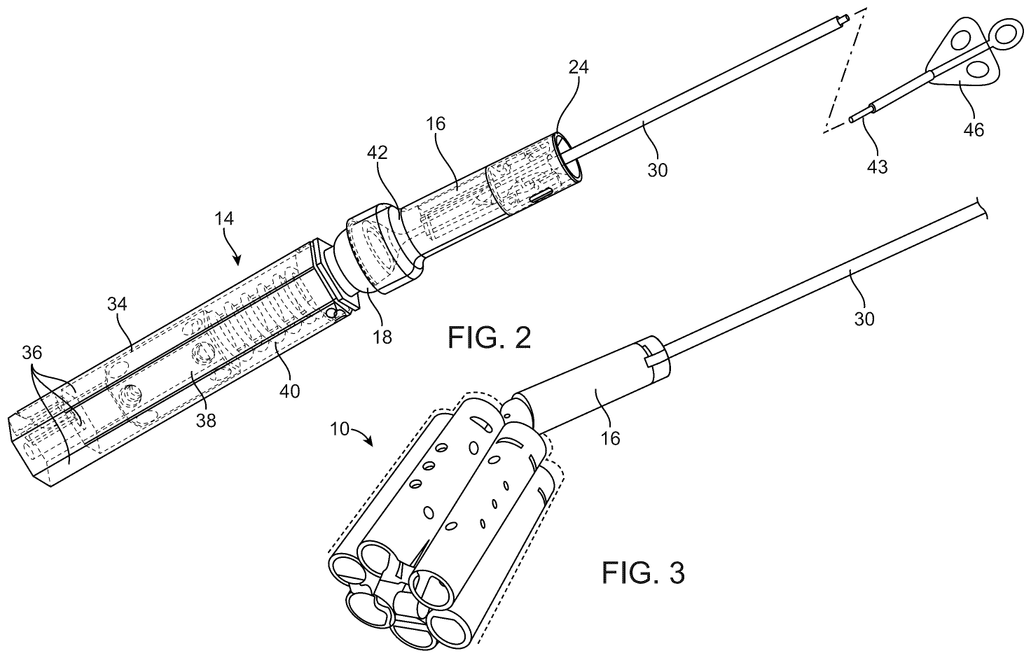

The hub 14 is typically connected to a manipulator segment 16 which is configured to detachably engage a support/release tool 30, as shown in FIGS. 2 and 3. In preferred examples, a distal end of the manipulator segment 16 is attached to a proximal end of the hub 14 by a universal joint 18. The freedom of movement afforded by the universal joint 18 facilitates engagement and attachment of the support/release tool 30 which is introduced through an open proximal end 24 of the manipulator segment 16.

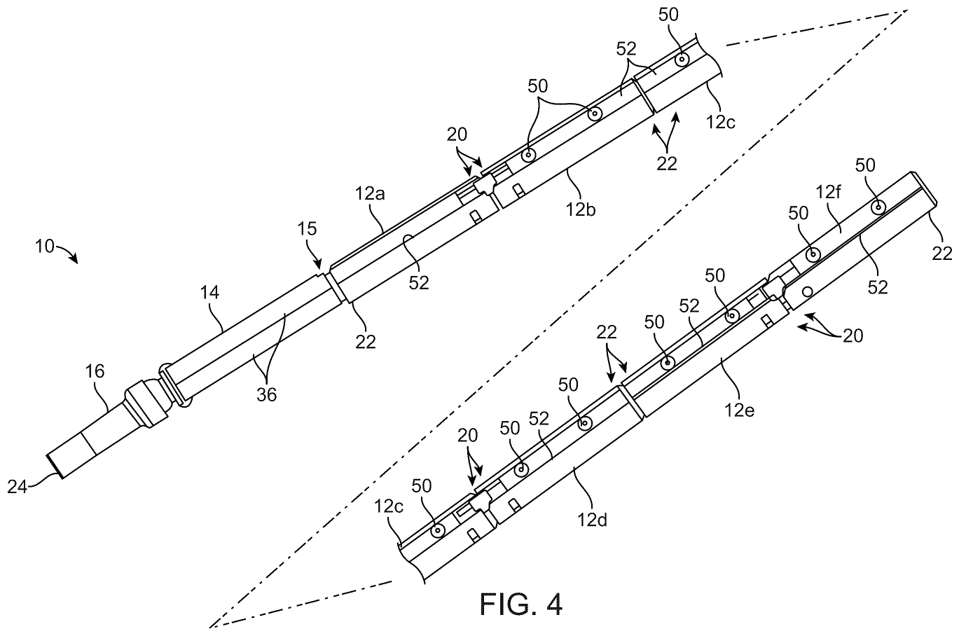

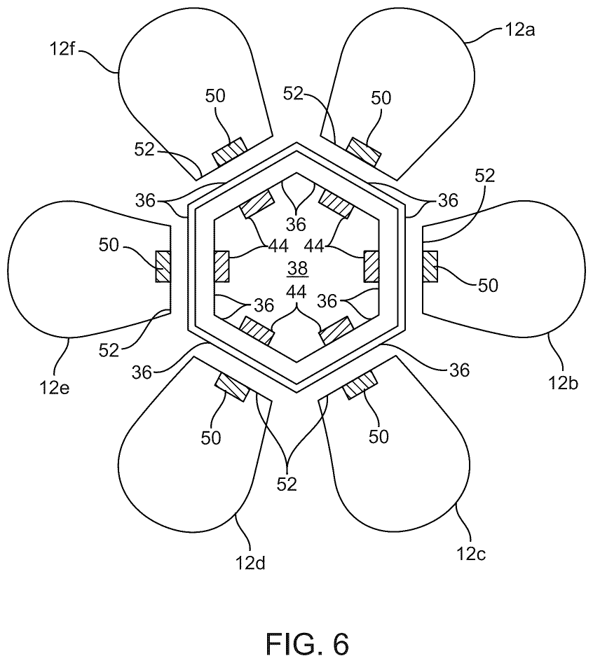

The hub 14 typically has a tubular body 34 with a periphery comprising flat faces or “facets” 36 which engage individual tool carrier 12 as they are brought into alignment with the hub. The hub 14 also carries a magnetic shuttle 38 which is secured on a distal side to a spring 40 which in turn is fixed to a closed distal end of the tubular body 34, allowing the shuttle to translate within the hub interior under biasing from the spring. As will be described in more detail below, the magnetic shuttle 38 carries individual magnets 44 (FIG. 6) which can align with magnets 50 (FIG. 4) on the individual tool carriers 12 to help stabilize the tool carrier array as tools are removed from and returned to the carriers.

A rod 43 in the support/release tool 30 couples to a cable 42 in the manipulator segment 16 when the tool 30 is attached to the manipulator segment through the open end 24, as shown in FIG. 2. The rod 43 may be proximally retracted using a three-ring actuator 46 on tool 30 to draw the cable 42 proximally, compressing spring 40 to align the magnets 44 with the magnets 50 on the individual carriers 12. Alignment of the magnets will close the cluster of tool carriers 12 from a relatively loose configuration, as shown in broken line in FIG. 3, to a tighter cluster, as shown in full line in FIG. 3.

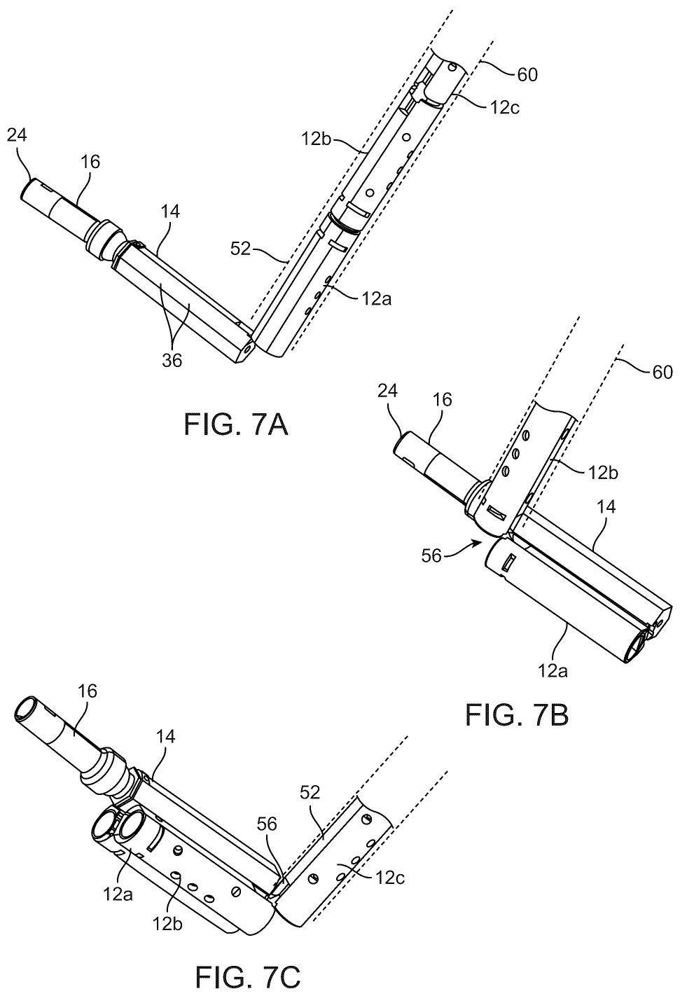

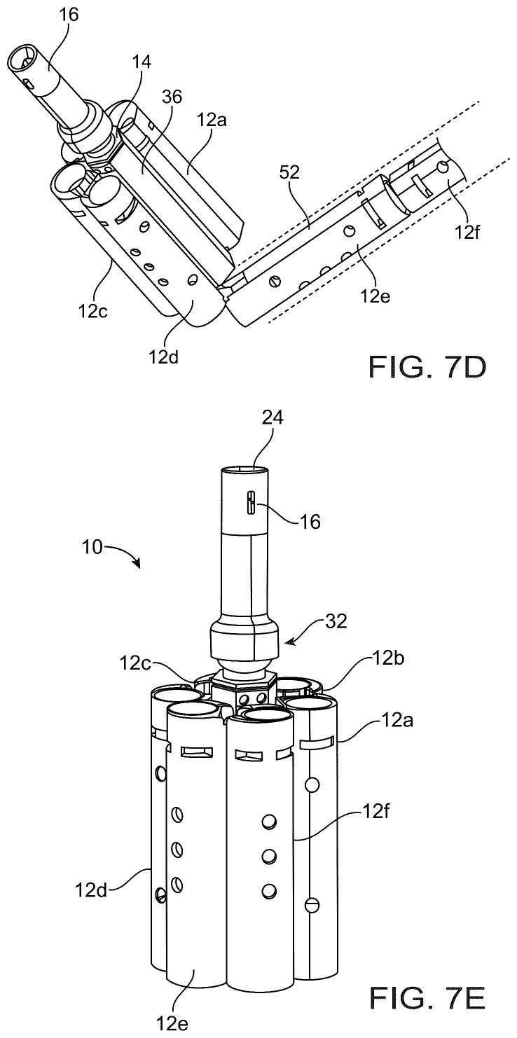

The tool carrier array 10 is shown in a straightened or elongated configuration suitable for delivery to a patient in FIG. 4. The carrier array is typically held within a delivery tube 60 (FIGS. 7A-7E) which radially constrains and straightens the individual tool carriers 12. Adjacent pairs of tool carriers 12 have articulated connections, typically defined by spring elements, that cause the adjacent tool carriers to fold against each other when released from constraint. For example, a bottom 15 of the hub 14 may be connected to the bottom 22 of the first tool carrier 12a. The top 20 of the first tool 12a, in turn, may connect to the top 20 of the second tool carrier 12b. The remaining tool carriers are linked similarly, allowing the structure to fold from the straightened configuration illustrated in FIG. 4 to the clustered configuration shown in FIG. 1.

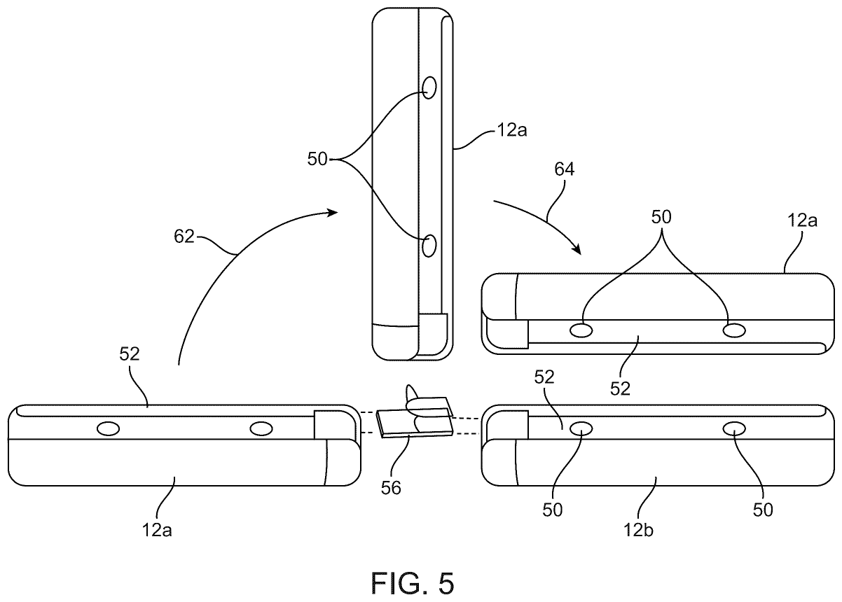

Referring now to FIG. 5, adjacent tool carriers 12a and 12b are joined by a flat spring 56 which is pre-formed into a U-shape, as shown in broken line. The flat spring 56 will be in its flattened configuration (full line) when the tool carriers 12a and 12b are axially aligned or otherwise elongated, as shown at the bottom of FIG. 5. When released from constraint, the spring 56 will revert to its U-shape, causing the tool carriers 12a and 12b to fold, as indicated by arrows 62 and 64.

Referring now to FIG. 6, the tool carriers 12a-12f will typically have at least one flat surface 52 configured to engage the flat surfaces 36 on the hub 14. Magnets 50 on the flat surface of 52 must be oriented toward the magnets 44 of the magnetic shuttle 38. When in close alignment, attraction between magnets 44 and magnets 50 will draw the tool carriers 12 to a tighter cluster. When the actuator 46 of the support/release tool 30 is used to translate the magnetic shuttle 38 and reposition the magnets out of alignment, the magnetic attraction will be reduced, allowing the individual tool carriers 12 a greater degree of freedom which may be necessary for withdrawal of the carriers and can also be useful when accessing surgical tool tips held in an individual carrier.

Referring now to FIGS. 7A-7E, delivery of the tool carrier array 10 to a thoracic, abdominal, or other patient body cavity will be described. The tool carrier array 10 is initially constrained in its straightened, elongated configuration in a tubular constraint 60. As shown in FIG. 7A, the hub 14 and manipulator segment 16 are initially advanced in a distal direction from the tubular constraint 60, allowing the spring connecting other the first tool carrier 12a to the hub 14 to fold the hub into the positions shown in FIG. 7B. As the tool carrier array 10 continues to be distally advanced from the tubular constraint 60, successive tool carriers 12b and 12c will be released and allowed to self-fold, as shown in FIGS. 7C and 7D. After the last tool carrier 12f is released from constraint, the tool carrier array 10 will assume the cluster array configuration shown in FIG. 7E.

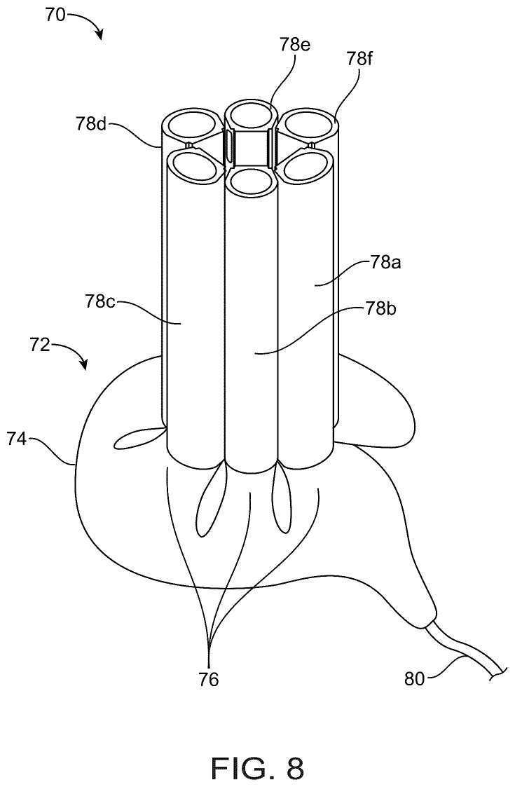

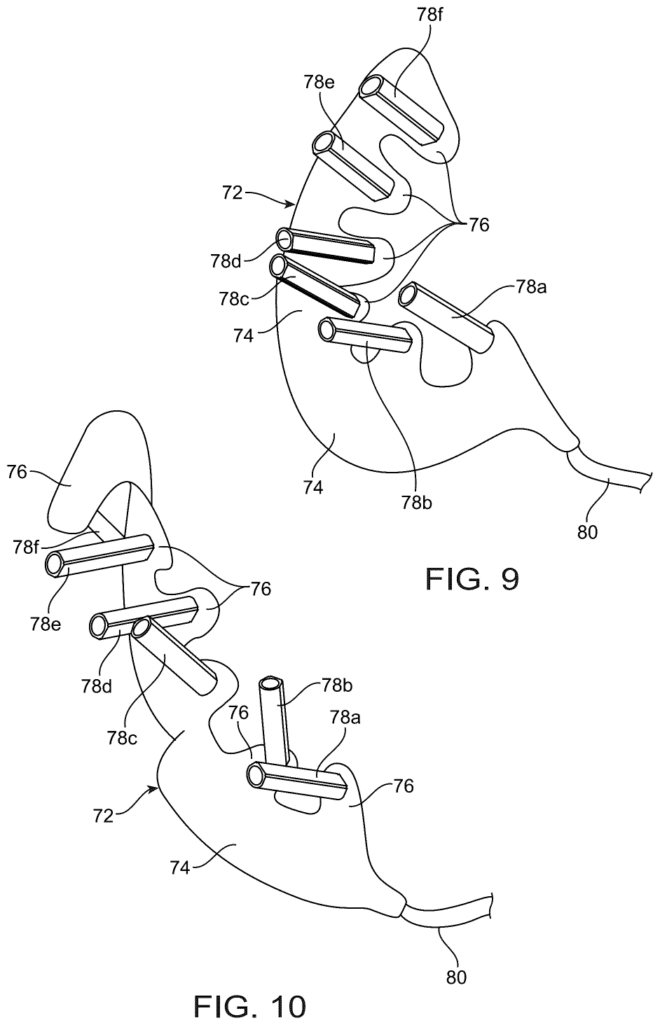

Referring now to FIGS. 8-10, an alternative embodiment of a tool carrier array 70 comprises an inflatable structure 72 for supporting a plurality of tool carriers 78. The tool carrier array 70 typically includes a C-shaped outer inflatable body 74, which forms a “backbone” of the structure, and plurality of spokes or fingers which project radially inwardly from the C-shaped inflatable body 74 when the inflatable support structure is fully inflated, as shown in FIG. 8. An inflation line 80 is provided to allow inflation after the tool carrier array has been placed in the patient. The spokes or fingers 76 each support an individual tool carrier 78. When uninflated, as shown in FIGS. 9 and 10, the inflatable structure 72 can be straightened or elongated, for delivery and removal. Typically, the dimensions of the inflatable structure 72 and the tool carriers 78 are chosen to provide sufficient clearance between adjacent tool carriers to allow the tool carriers to axially align as tension is applied to the ends of the deflated inflatable structure 72. Such alignment allows the tool carriers to lie end-to-end, minimizing the profile of the carrier array 70 and facilitating delivery and removal.

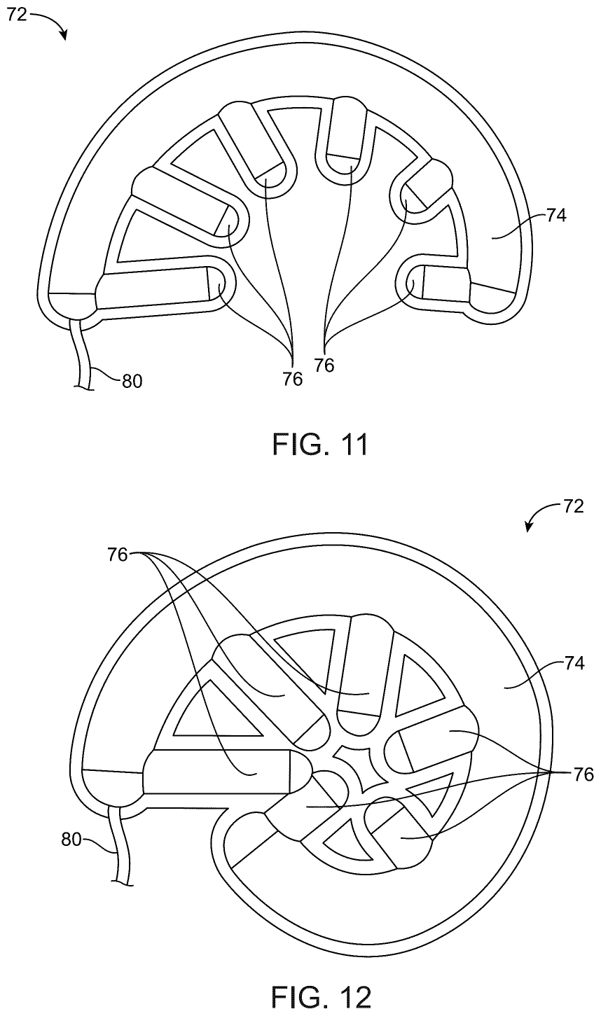

Referring now to FIGS. 11 and 12, a preferred design of inflatable support structure 72 is shown, uninflated in FIG. 11 and fully inflated in FIG. 12. The lengths of the individual spokes or fingers 76 vary allowing the degree of curvature to increase toward one end of the inflated structure, is best seen in FIG. 12.

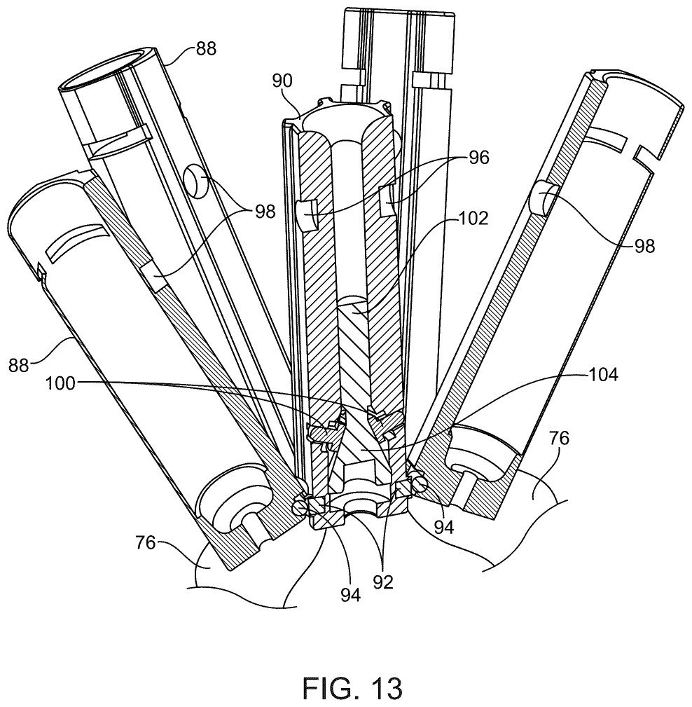

While the inflatable supports 72 in FIGS. 8-11 may be used without further structure, it will often be desirable to provide additional supporting features or components. For example, as shown in FIG. 13, a hub 90 may be positioned at a center region of the array of tool carriers 88 while the tool carriers are supported by the inflated structure 72. The hub 90 will not be mechanically linked to the carriers 88, as was the case in the prior spring-linked embodiment and will typically provide only magnetic stabilization to improve alignment and clustering of the tool carriers 88 while the tool carriers are supported by the inflatable structure 72.

As seen in FIG. 13, the hub will have a plurality of magnets 92 at its base end which can align with magnets 94 at the bottoms of the tool carriers 88. The magnet pairs 92/94 can act as a virtual hinge, allowing the individual tool carriers 88 to pivot outwardly as shown in FIG. 13. In addition or alternatively, magnets 96 may be provided on the body of the hub 90 to align with magnets 98 on upper portions of the tool carriers 88. The magnetic pairs 96/98 will improve clustering of the cluster while still allowing the pivoting of the tool carriers when removing and returning surgical tool tips.

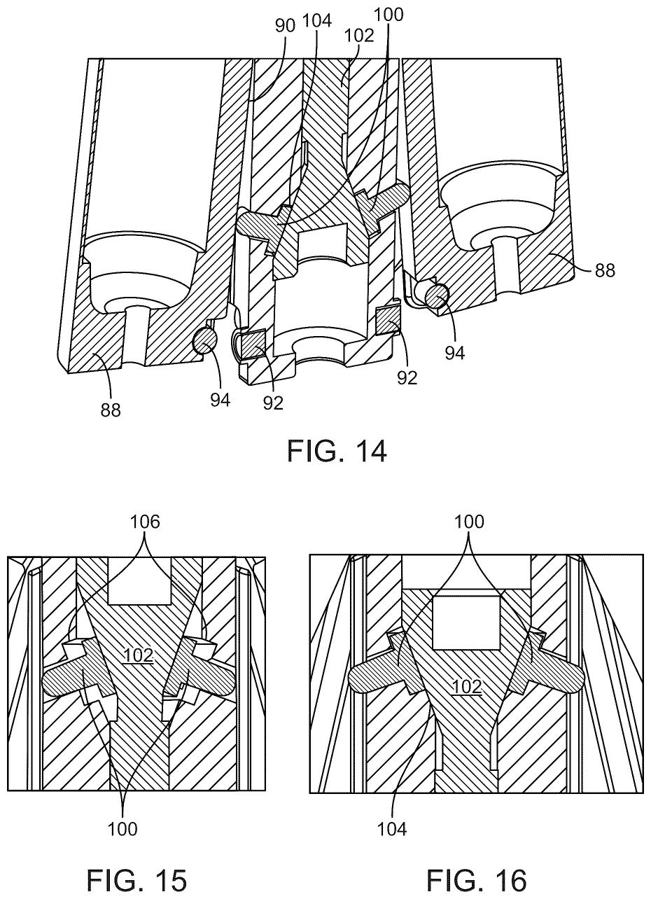

Usually, the hub 90 will have to be disengaged from the tool array 70 before the array can be removed from the patient. To facilitate such disengagement, a plurality of pins 100 may be located near the bottom of hub 90, as seen in FIGS. 14-16. The pins 100 are initially retracted into receptacles 106 formed in the hub body. A piston 102 is mounted to reciprocate in a central passage in the hub and has a conical surface 104. When the piston 102 is in its upward position, as shown in FIG. 15, the pins 100 remain retracted in the outer surface of the hub 90. By pushing the piston 100 downwardly, however, the conical surface 104 engages the pins 100, causing the pins to move outwardly, as shown in FIG. 16. Such outward movement of the pins 100 will engage and displace the lower ends the individual tool carriers 88, disrupting the magnetic coupling between the tool carriers 88 and the hub 90, as shown in FIG. 14.

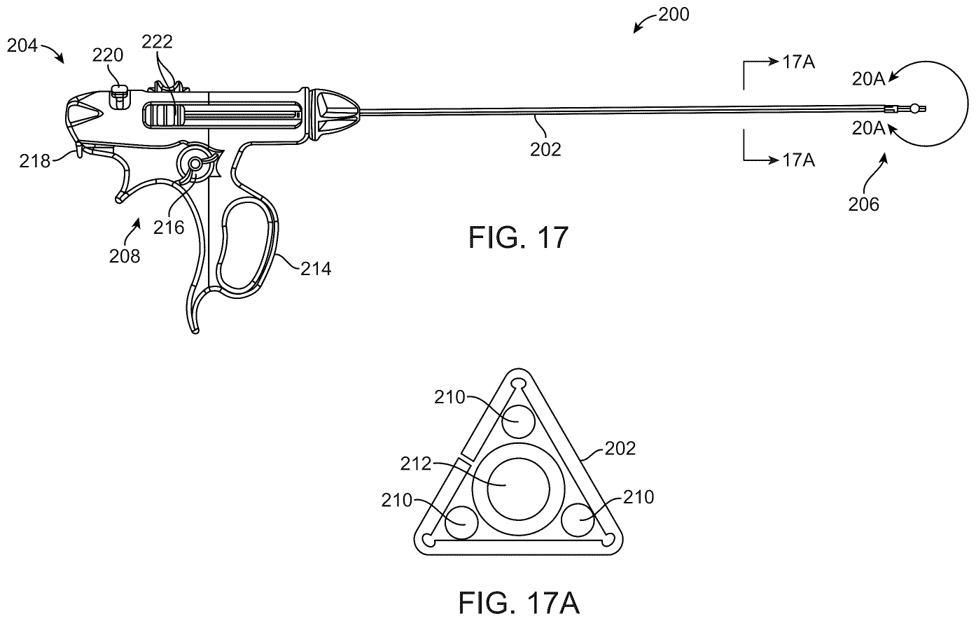

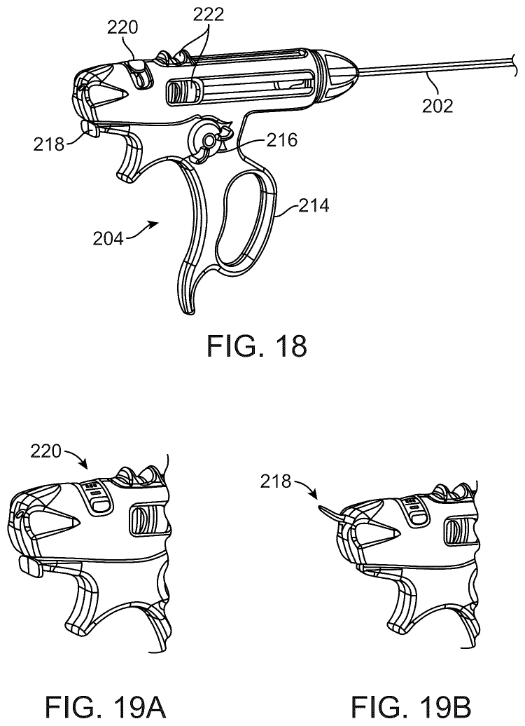

FIGS. 17 and 18 illustrate a tool tip cassette deployment and support tool 200 having a shaft 202 with a proximal end 204, a distal end 206, and a deployment handle 208 at the proximal end. The shaft 202 has a triangular cross-section, as seen in FIG. 17A, with three passages 210 at corners of the triangle for carrying pushers 234, as described below. A central passage 212 is configured to slidably receives a central member, as described below. The deployment handle 208 includes a hand grip 214, a mode selector 216, a trocar flag 218, a trocar lock-out 220, and three sliders 222 (only two of which are visible in FIG. 18).

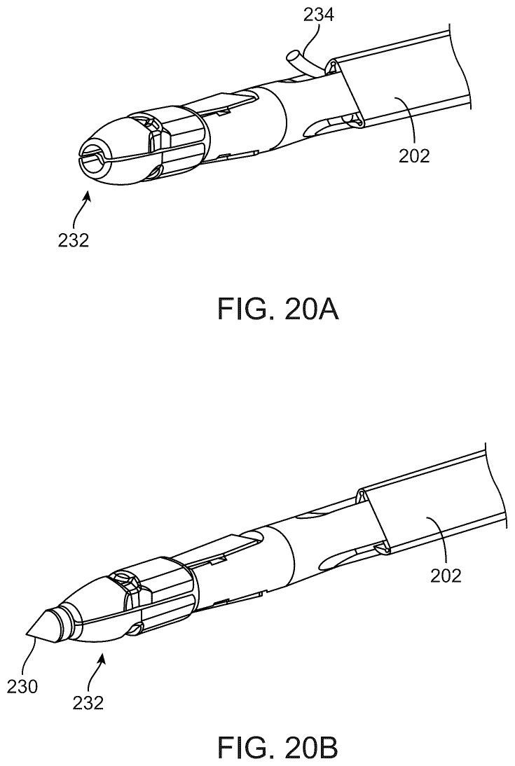

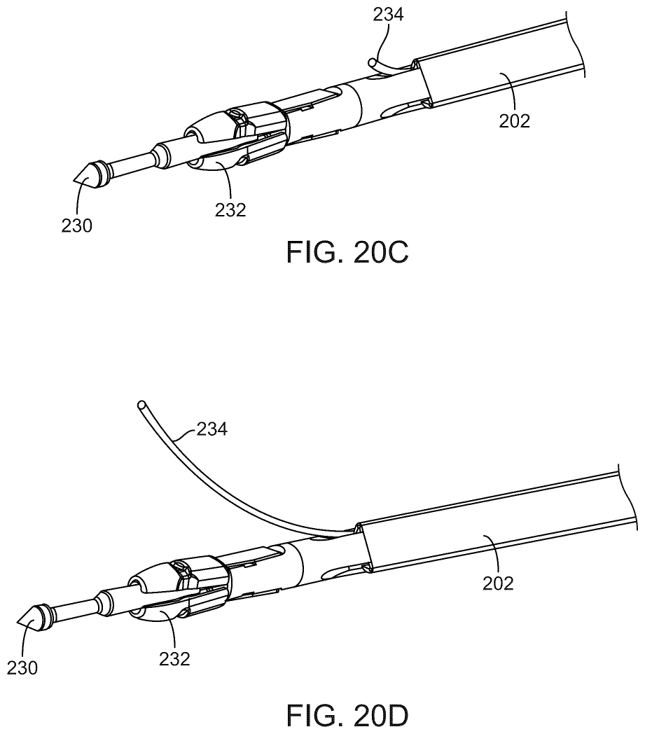

As shown in FIGS. 17, 18, and 20A, the tool tip cassette deployment and support tool 200 is in its “safe” mode with piercing tip 230 retracted. To enter the “piercing” mode, the trocar lock-out 220 is moved to the side to retract pusher 234, as shown in FIGS. 19A and 20A, and the trocar flag 218 flipped up, as shown in FIG. 19B, to advance the piercing tip 230 through a clam-shell type nose cone 232, as shown in FIG. 20B. The mode selector 216 may then be used to axially position the piercing tip 230 in order to capture and release the tool tip cassette 200 and the sliders 222 used to advance and retract the pushers 234, as described in more detail below.

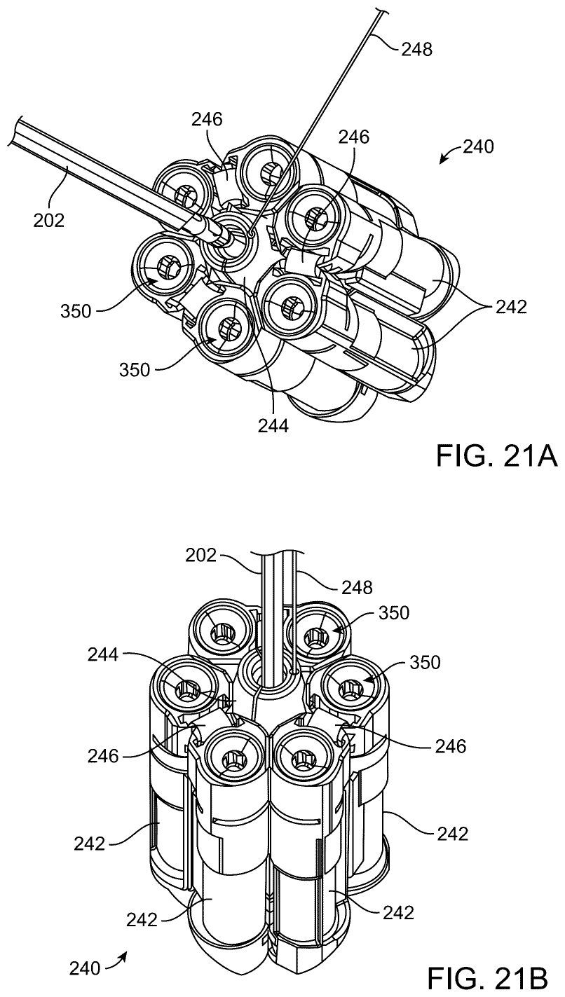

FIGS. 21A and 21B show a tool tip cassette 240 with tool carriers 242 in a parallel cluster configuration as it is being engaged and captured by the distal end of the tool tip cassette deployment and support tool 200 of FIG. 17. The structure of the tool tip cassette 240 is generally as described above where the tool carriers 242 are held on a hub 244 and joined by springs 246. A tether 248 is provided to suspend and manipulate the tool tip cassette 240 as it is engaged and captured by the of the tool tip cassette deployment and support tool 200.

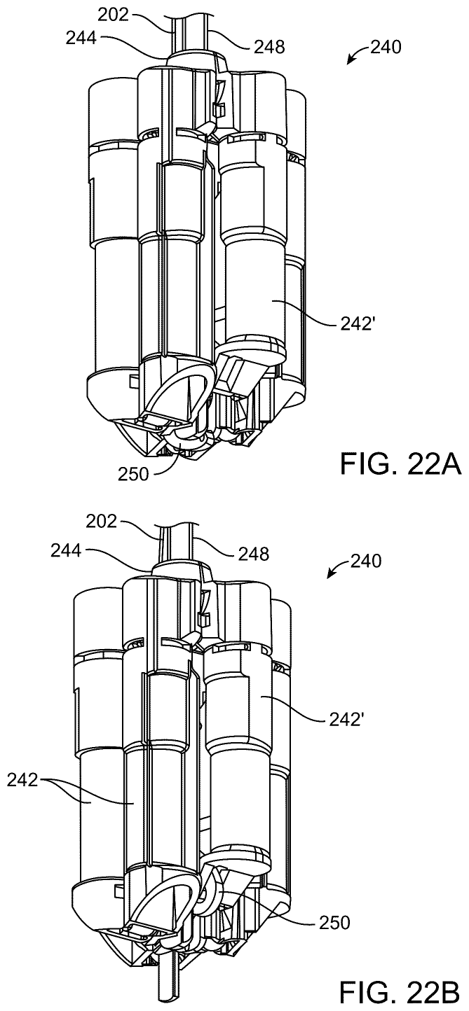

As described with reference to previous embodiments, the tool carriers of the present invention may be magnetically coupled to the hub after cluster formation. The tool tip cassette 240 now being described includes a mechanical latch 250, typically formed as a hook, to connect a free distal tool carrier 242′ of the cluster to the hub 244 (i.e., the tool carrier in the linked chain furthest from the hub 244). As shown in FIG. 22A, the latch 250 is open, i.e. disengaged from the free distal tool carrier 242′. The latch is shown in its closed or engage configuration in FIG. 22B.

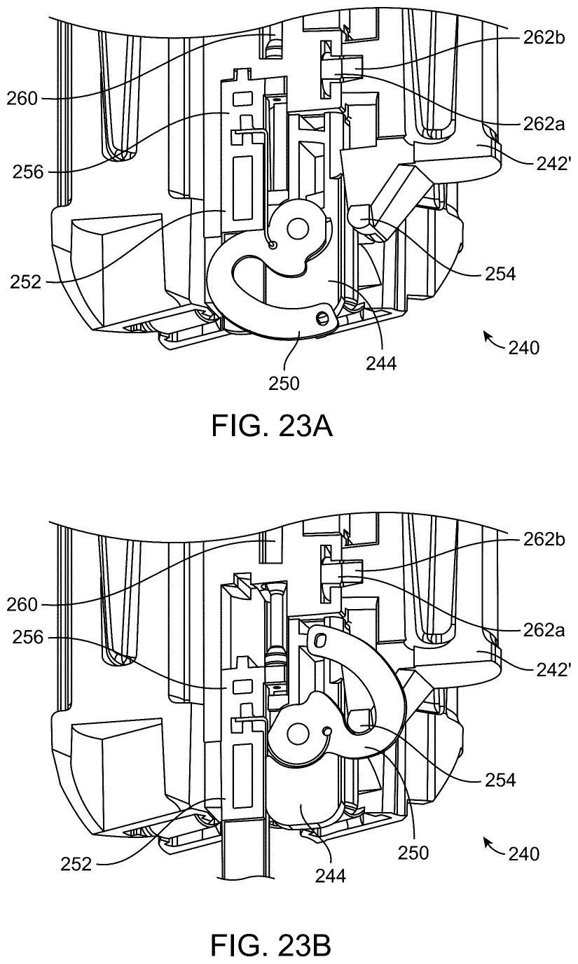

FIGS. 23A and 23B provide cross-sectional views of the lower portion of the tool tip cassette 240. The hook 250 is pivotally attached to a wall of the hub 244 and configured to be engaged by a sliding shuttle 252. A trocar tip 232 is advanced from the distal end of the shaft 202 to advance a shuttle linkage 256 after the tool tip cassette deployment and support tool 200 has captured the tool tip cassette 240, causing the shuttle 252 to engage and pivot the hook 250 from the position shown in FIG. 23A to that shown in FIG. 23B where it captures a hook catch 254 on the free distal tool carrier 242′. Prior to engaging the hook 250, alignment magnets 262a and 262b preposition free distal tool carrier 242′ relative to the hub 244. Magnet 262a is on the hub 244 and magnet 262b is on the free distal tool carrier 242′.

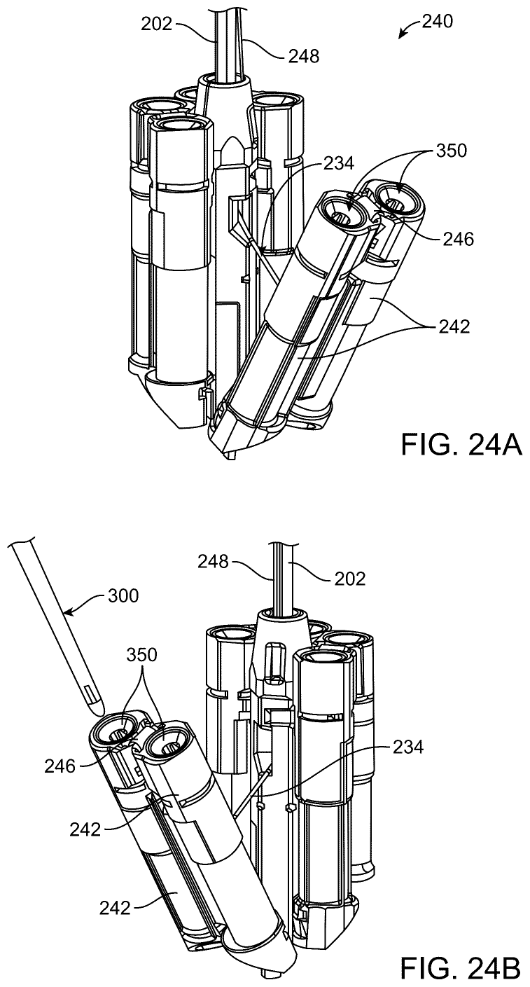

FIGS. 24A and 24B are detailed views showing the pusher 234 radially deflecting tool carriers 242 from the tool carrier cassette cluster 240 to facilitate removal of surgical tools from the tool carriers. As described describe previously, sliders 222 on handle 208 are used to selectively advance each of the three pushers 234, and each one of the pushers 234, in turn, is configured to engage a pair of tool carriers 242 joined by a spring 246 in order to deflect that pair of tool carriers radially outwardly. In that position, the surgical tool carrier can be engaged by a surgical tool driver 300 as described in detail below.

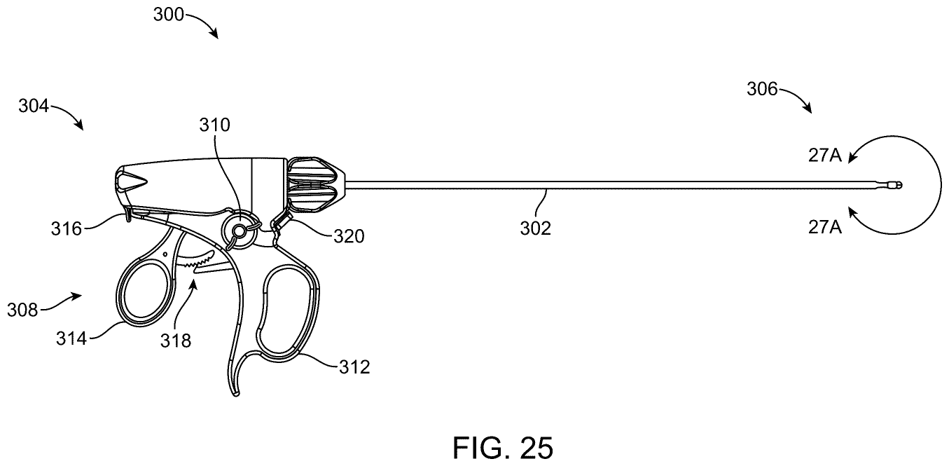

The surgical tool driver 300 is illustrated FIG. 25 and includes a shaft 302 having a proximal end 304, a distal end 306, and a deployment handle 308 at the proximal end. The deployment handle 308 further includes a mode selector 310, a fixed grip 312, a thumb lever 314, and a trocar flag 316. A ratchet mechanism 318 is coupled to a ratchet selector 320 to control operation of the thumb lever 314.

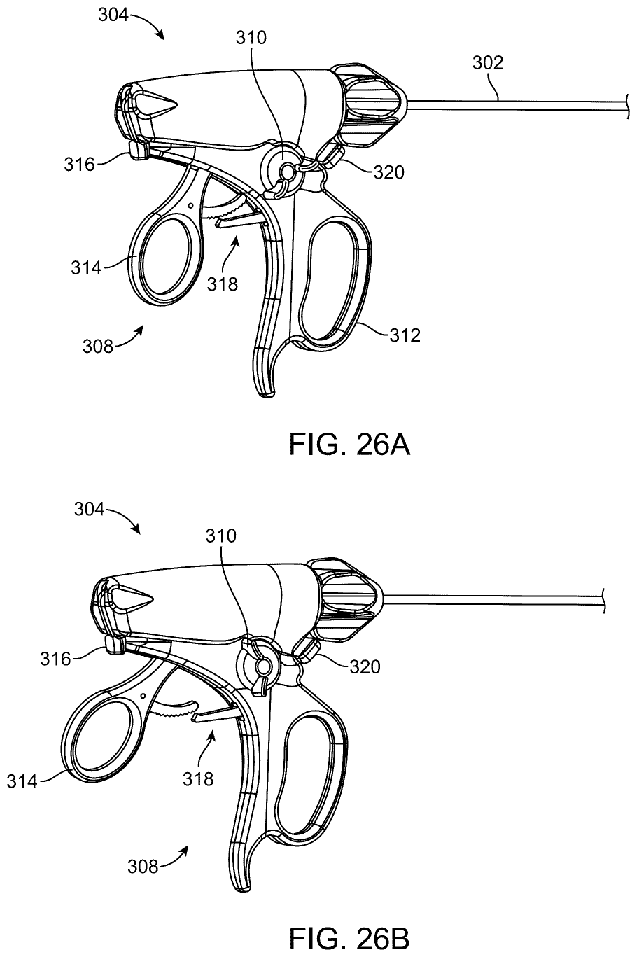

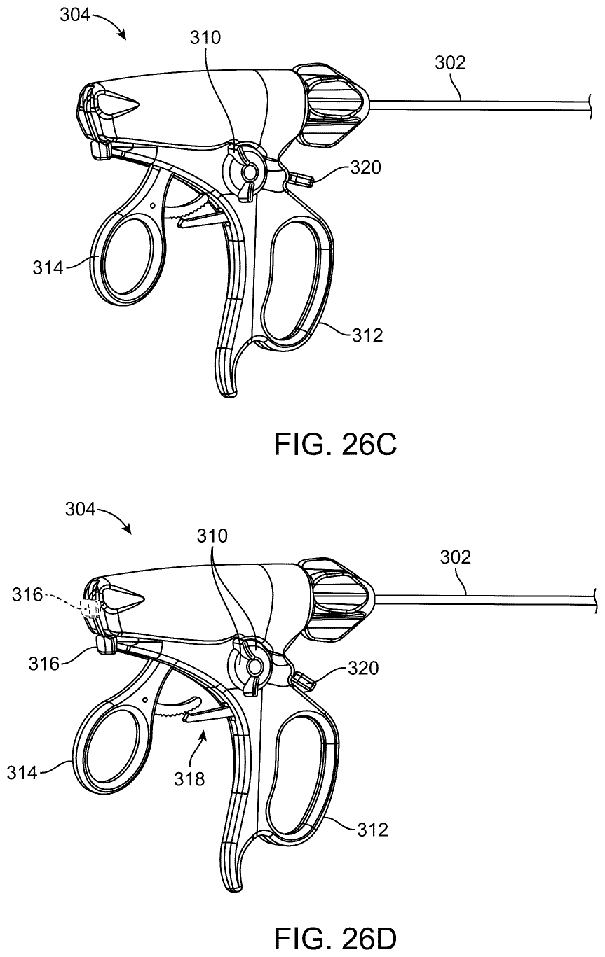

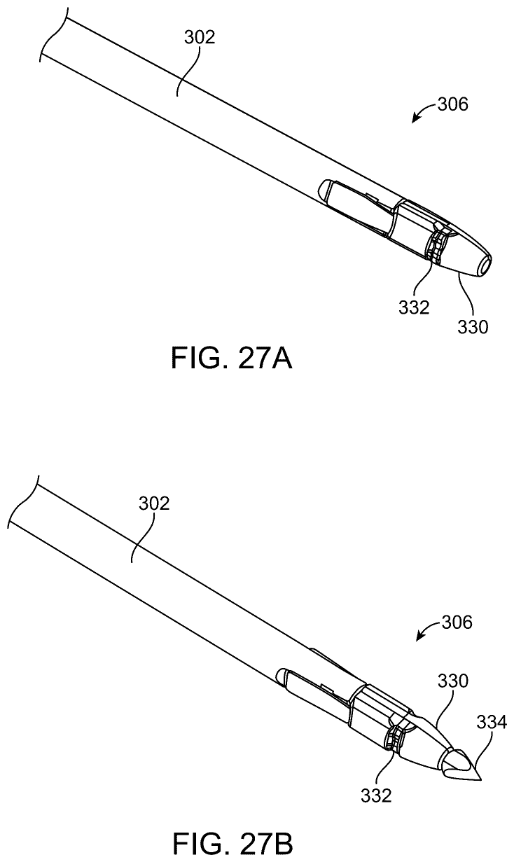

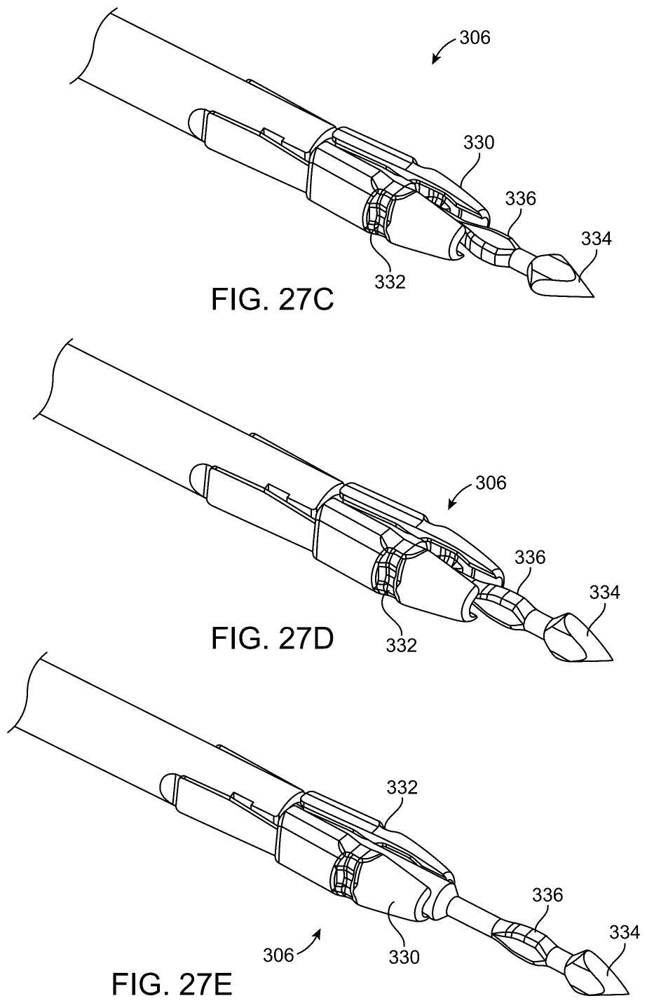

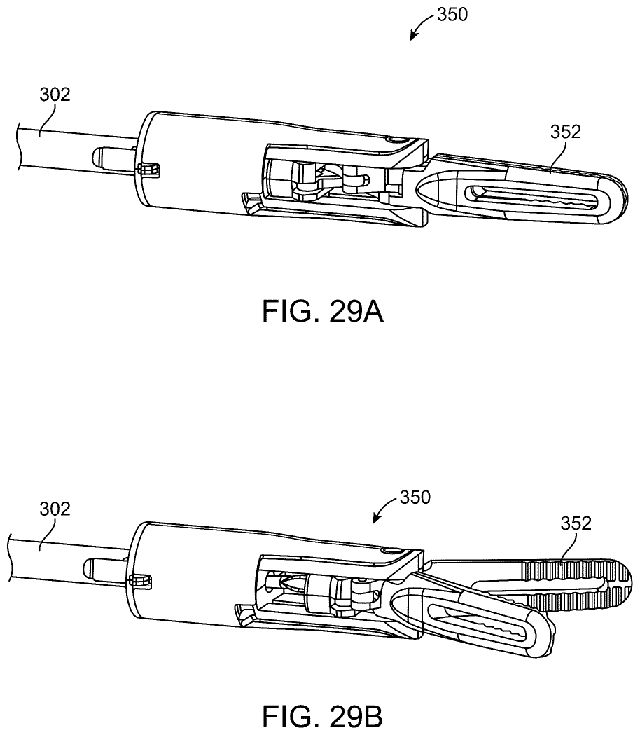

As shown in FIG. 26A, the mode selector 310 is in a first position where the surgical tool driver 300 is in a “safe” mode where all functions of the driver are disabled. By rotating the mode selector 310, as shown in FIG. 26B, the ratchet selector 320 and the thumb lever 314 are enabled in a “tool actuation” mode. By rotating the mode selector 310 to the “tool actuation mode,” the piercing tip 334 is also advanced to the state shown in FIG. 27C and then rotated as shown in FIG. 27D. This action docks the distal end 306 of the surgical tool driver 300 to the tool tip 350, e.g., by wedging nose cone 330 into a cavity within a shank or shaft portion of the tool tip 350 as well as advancing and rotating to lock the tip within the shuttle that actuates the tool tip jaws 352 (FIGS. 29A and 29B).

In the tool actuation mode, a piercing tip 334 may be distantly advanced and rotated, as shown in FIGS. 27B to 27E. The thumb lever 314 may be further distally advanced, as shown in FIGS. 27C to 27E, to actuated tool, for example open and close jaws 352 in a surgical tool tip 350, as shown in FIGS. 29A and 29B.

The ratchet 318 and ratchet selector 320 are engaged and momentarily disengaged as shown in FIGS. 26C and 26D, respectively. When the ratchet 318 is engaged, the thumb lever 314 can only be closed. The ratchet selector 320 is spring-loaded to automatically return from the momentary off position (FIG. 26D) to the on position (FIG. 26C) when the selector is no longer pressed.

FIGS. 27A to 27E are enlarged views of the distal end 306 of the shaft 302 of the tool driver 300 of FIG. 25 shown in different configurations. As shown in FIG. 27A, the surgical tool driver 300 is in a safe mode with the piercing tip 334 retracted behind the nose cone 330. The piercing tip 334 may be advanced using the thumb lever 314 of the handle 308 as previously described, as shown in FIG. 27B. To advance the piercing tip 334, the trocar flag 316 must be in a “flipped up” position, as shown in broken line in FIGS. 26D and 27D. With the piercing tip 334 in the position shown in FIG. 27B, the tool driver may be percutaneously introduced through the patient's abdomen without use of a separate cannula.

As shown in FIGS. 27D to 27E, the thumb lever 314 of the handle 308 may be further manipulated to advance the piercing tip 334 and in order to operate those surgical tool tips once they've been loaded onto the surgical tool driver 300.

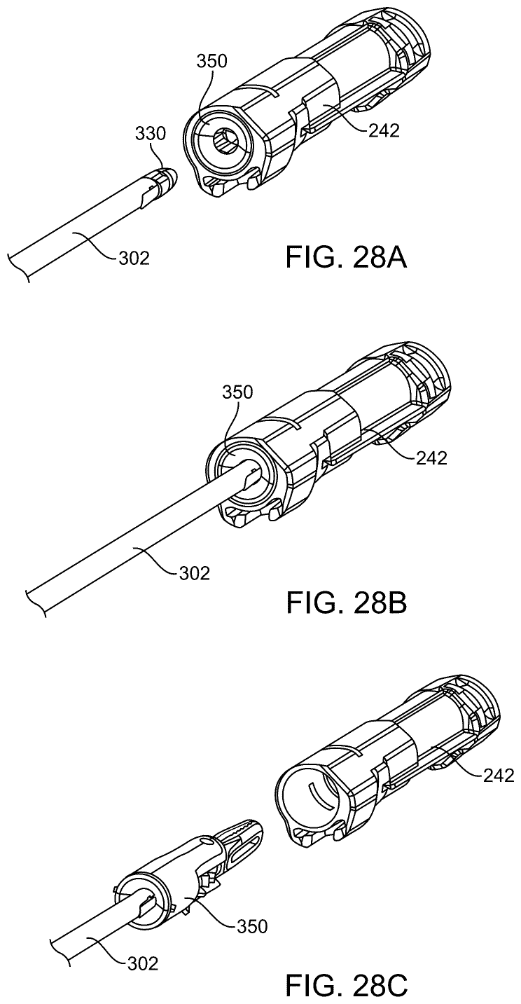

FIGS. 28A to 28C show the nose cone 330 of the surgical tool driver 300 engaging and removing a surgical tool 350 from a tool carrier 242.

FIGS. 29A and 29B show the surgical tool tip 350 being actuated by the surgical tool driver 300. For example, jaws 352 of a surgical tool tip 350 may be actuated by advancing and retracting the piercing tip 334 of the driver 300 as shown in FIGS. 27D (jaws closed) and 27E (jaws open).

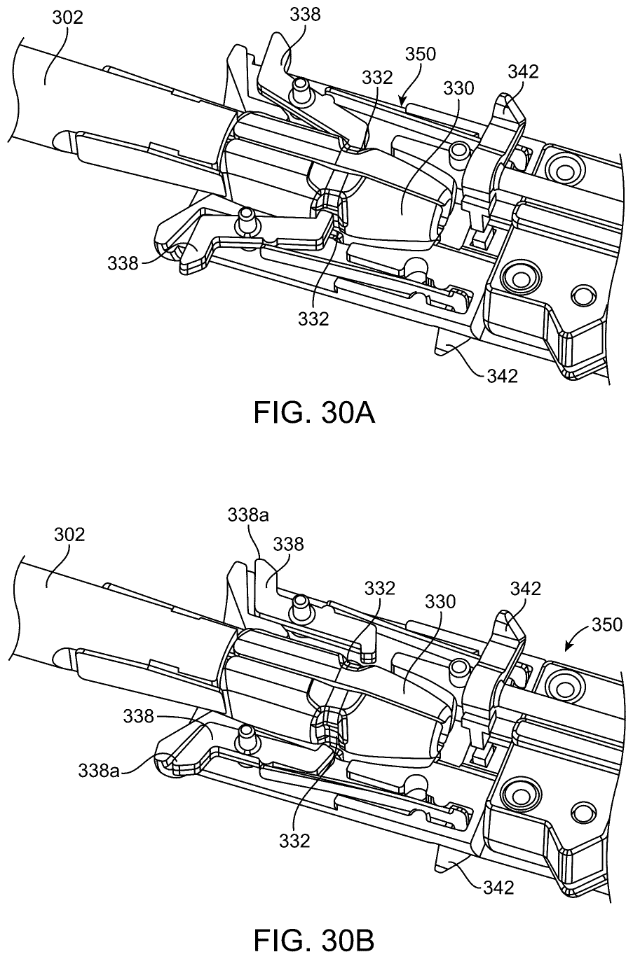

FIGS. 30A and 30B show a safety lock mechanism which prevents the surgical tool from being accidentally dropped from the tool driver when the surgical tool is outside the tool carrier. When the surgical tool tip 350 is removed from the tool carrier 242 (not shown), a pair of spring-loaded nose cone catches 338 engage a pair of catch grooves 332 on the nose cone 330 (FIG. 30A). These catches prevent accidental disengagement of the surgical tool tip 350 from the shaft 302 of the surgical tool driver 300. When the surgical tool tip 350 is returned to the tool carrier, outer ends 338a of each nose cone catch 338 are depressed inwardly, removing the opposite ends of the catches from the catch grooves 332 on the nose cone 330 (FIG. 30B). Thus, the tool driver 330 can be withdrawn from the surgical tool tip 350 once the tip is placed back into the tool carrier.

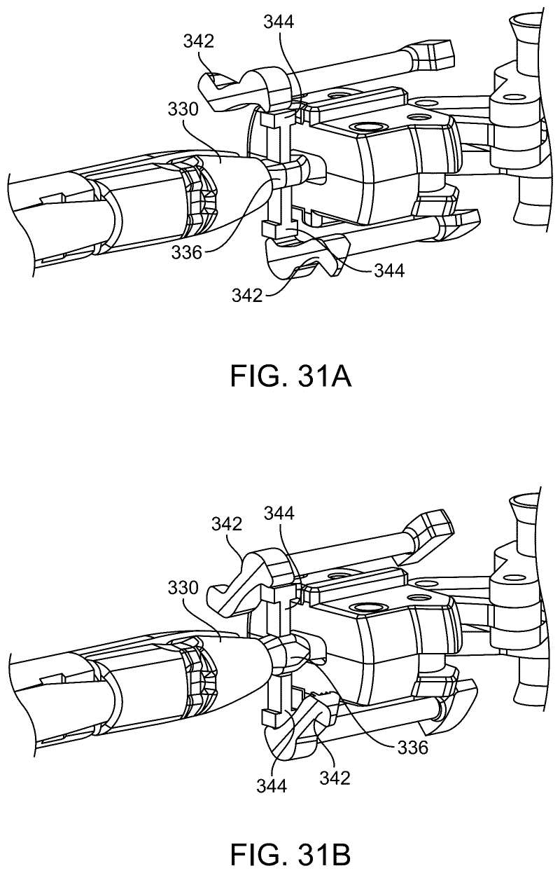

FIGS. 31A and 31B show a tool retention mechanism comprising a pair of tool retention tabs 342 configured to hold the surgical tool 350 in the tool carrier (not shown) when not in use. The tool retention mechanism may be actuated by the surgical tool driver 300 to allow the tool to be removed from the carrier when the particular tool is needed for a procedure. After the nose cone 330 of the surgical tool driver 300 has been introduced into the open end of the surgical tool tip 350, the cam lobe 336 is advanced to a position between a pair of extensions 344. Outer tips of the tool retention tabs 342 are in a radially outward position and engage slots (not shown) in the wall of a base of the surgical tool tip to prevent accidental disengagement of the tool tip from the surgical tool carrier. By rotating the cam lobe 336 by 90°, as shown in FIG. 31B, extensions 344 are caused to move radially outwardly to engage and rotate tool retention tabs 342 causing the free ends of the tabs to move radially inwardly and disengage from the slots. Once the tool retention tabs 342 are disengaged from the slots, the surgical tool tip may be removed from the tool carrier.

While the previous embodiments of both the tool tip cassette deployment and support tool 200 and the surgical tool driver 300 employ split nose cones to allow advancement of tissue-piercing tip, in other embodiments (not illustrated) an auger tip having a retractable sharp could be used without a splittable nose cone or other protector. Such auger tips could provide easier tissue penetration for percutaneous access (drilling vs piercing/stretching) and potentially allow more robust connection to the tools and cassette.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A tool tip cassette comprising:
a deployment mechanism;
a plurality of tool carriers coupled to the deployment mechanism, wherein each tool carrier has a tool access end and a base end; and
wherein the deployment mechanism is adapted to selectively reconfigure the plurality of tool carriers from a linear arrangement into a parallel cluster.

2. The tool tip cassette as in claim 1, further comprising surgical working tips removably placed in at least some of the plurality of tool carriers.

3. The tool tip cassette as in claim 1, wherein the tool carriers comprise tubular bodies, typically cylindrical bodies.

4. The tool tip cassette as in claim 1, further comprising a hub configured to occupy a center region of the parallel cluster of tool carriers when said tool carriers are unconstrained.

5. The tool tip cassette as in claim 4, further comprising a manipulator segment attached to the hub, wherein at least one of the tool carriers and the hub each comprise at least one magnetic element, wherein the at least one magnetic element on the hub is configured to attract the magnetic element on the at least one tool carriers to hold the plurality of tool carriers in the in the parallel cluster configuration while allowing limited displacement to facilitate removal and return of tool tips from the tool carriers.

6. The tool tip cassette as in claim 5, wherein the hub comprises axial facets about its periphery, with each facet being configured to engage a first flat surface on one of the tool carriers.

7. The tool system as in claim 6, wherein the plurality of tool carriers comprise one of (1) three tool carriers with axial facets having a 120° spacing, (2) four tool carriers with axial facets having a 90° spacing, (3) five tool carriers with axial facets having a 72° spacing, (4) six tool carriers with axial facets having a 60° spacing, (5) seven tool carriers with axial facts having a 51.4° spacing, and (6) eight tool carriers with spring axial facets having a 45° spacing.

8. The tool tip cassette as in claim 7 wherein each tool carrier comprises two additional flat surfaces arranges symmetrically on each side first flat surface and configured to nest with adjacent tool carriers when the tool carriers are in the parallel cluster formation.

9. The tool tip cassette as in claim 1, wherein the deployment mechanism comprises spring elements which attach the base ends of individual tool carriers to the tool access ends of adjacent tool carriers, wherein the spring elements on adjacent tool carriers are circumferentially offset so that the spring elements cause the tool carriers to fold into the parallel cluster when unconstrained, wherein the springs members comprise two legs having a U-shape when unconstrained, wherein a first leg is attached to a base end of one tool carrier and a second leg is attached to a base end of an adjacent tool carrier.

10. The tool tip cassette as in claim 4, wherein the hub is attached to the base end of the first tool carrier by a U-shaped spring configured so that the plurality of tool carriers fold into the parallel cluster about a periphery of the shuttle segment when released from constrained.

11. The tool tip cassette as in claim 1, further comprising an over tube having a passage for straightening and delivering the plurality of tool carriers to a surgical site.

12. The tool tip cassette as in claim 1, wherein the deployment mechanism comprises an inflatable structure, wherein the plurality of tool carriers can align axially when the inflatable structure is deflated and will arrange into the parallel cluster when the inflatable structure is inflated.

13. The tool tip cassette as in claim 12, wherein the inflatable structure comprises a ring or disc.

14. The tool tip cassette as in claim 13, wherein the inflatable structure comprises an open ring with spokes and wherein the tool tip holders are attached to radially inward ends of the spokes.

15. The tool tip cassette as in claim 4, wherein individual tool carriers or groups of tools carriers are configured to be displaced away from the hub to facilitate access to a tool carried by the tool carrier.

16. The tool tip cassette as in claim 15, wherein the individual tool carriers or groups of tools carriers are configured to be displaced away from the hub by engagement with a deployment mechanism, such as a pusher.

17. The tool tip cassette as in claim 4, further comprising a tether attached to the hub and configured to temporarily support the parallel cluster of tool carriers prior to capture by a tool tip cassette deployment and support tool and/or remove the tool tip cassette from a work site.

18. A tool tip cassette delivery system comprising:
the tool tip cassette of claim 5; and
a tool tip cassette deployment and support tool having a shaft with a proximal end and a distal end; and
a handle at the proximal end of the shaft;
wherein the distal end of the shaft is configured to detachably engage the hub of the tool tip cassette.

19. The tool tip cassette delivery system as in claim 18, wherein the deployment and support tool is configured to engage the manipulator segment and selectively align and misalign magnets in the hub with magnets in the tool the tool carriers of the tool tip cassette to form and release the parallel cluster arrangement.

20. The tool tip cassette delivery system as in claim 19, wherein the magnets within the hub are mounted to axially translate and the cassette manipulator comprises a pusher or puller to move the hub magnets in and out of alignment with the tool tip holder magnets.

21. The tool tip cassette delivery system as in claim 20, wherein the manipulator segment comprises a puller cable and the hub magnet is spring-mounted so that puller cable can apply tension to align the hub and tool tip holder magnets while the cassette manipulator remains attached to the hub.

22. The tool tip cassette delivery system as in claim 18, wherein the manipulator segment and the hub are connected via a universal joint.

23. The tool tip cassette delivery system as in claim 22, wherein the universal joint is located at the distal end of the manipulator segment and a proximal end of the hub.

24. The tool tip cassette delivery system as in claim 18, wherein the tool tip cassette deployment and support tool comprise a reciprocatable tip.

25. The tool tip cassette delivery system as in claim 24, wherein the reciprocatable tip is configured to pierce tissue when distally advanced.

26. The tool tip cassette delivery system as in claim 24, wherein the reciprocatable tip is configured to engage and release the tool tip cassette when said cassette in its parallel cluster arrangement.

27. The tool tip cassette delivery system as in claim 18, wherein the tool tip cassette deployment and support tool further comprise one or more pushers configured to displace individual tool carriers or groups of tools carriers away from the shaft to facilitate access to surgical tools carried by the tool carriers.

28. The tool tip cassette delivery system as in claim 18, further comprising at least one surgical tool driver comprising:
a shaft with a proximal end and a distal end; and
a handle at the proximal end of the shaft;
wherein the distal end of the shaft is configured to detachably engage and operate individual surgical tool tips carried by the tool tip cassette.

29. The tool tip cassette delivery system as in claim 28, wherein the at least one surgical tool driver comprises a reciprocatable tip at the distal end of the shaft.

30. The tool tip cassette delivery system as in claim 29, wherein the reciprocatable tip is configured to pierce tissue when distally advanced.

31. The tool tip cassette delivery system as in claim 29, wherein the reciprocatable tip is configured to detachably engage and operate the individual surgical tools.

32. The tool tip cassette delivery system as in claim 28, further comprising a first interlock configured to prevent disengagement of the surgical tool tip from the shaft when the tip is removed from the tool carrier.

33. The tool tip cassette delivery system of claim 28, further comprising a second interlock configured to prevent loss of the surgical tool tip from the tool carrier when the tool tip is not engaged with the tool driver.

34. The tool tip cassette delivery system as in claim 18, wherein each tool carrier comprises a magnetic element.

35. The tool tip cassette delivery system as in claim 18, wherein at least one of the tool carriers comprises a latch mechanism configured to selectively engage the hub to maintain all the tool carries in the cluster formation while allowing limited displacement to facilitate removal and return of tool tips from the tool carriers.

36. A method for delivering surgical tools to a surgical working space, said method comprising:
providing a tool tip cassette including a plurality of tool carriers joined end-to-end;
percutaneously introducing tool tip cassette to the surgical working site while the plurality of tool carriers is in a linearized arrangement;
reconfiguring the plurality of tool carriers from the linearized arrangement into a parallel cluster while in the surgical working space; and
retrieving and returning surgical tools from individual tool carriers while the plurality of tool carriers remains in the parallel cluster arrangement in the surgical working space.
